(19) United States
(12) Patent Application Publication
(10) Pub. No.: US 2025/0340263 A1
(43) Pub. Date: Nov. 6, 2025

(54) Dynamic Prosthetic Interface Biking Mount for Transfemoral Amputees

(71) Applicant: UNM Rainforest Innovations, Albuquerque, NM (US)

(72) Inventors: Christina Salas, Albuquerque, NM (US); Giovanni Cordova, Albuquerque, NM (US); Natalia McIver, Albuquerque, NM (US); Brandon Doehne, Albuquerque, NM (US); Evan Long, Albuquerque, NM (US); Melachi Sanchez, Albuquerque, NM (US); Jacob Whisenhunt, Albuquerque, NM (US); Emma White, Albuquerque, NM (US)

(73) Assignee: UNM Rainforest Innovations, Albuquerque, NM (US)

(21) Appl. No.: 19/197,937

(22) Filed: May 2, 2025

Related U.S. Application Data
(60) Provisional application No. 63/641,933, filed on May 2, 2024.

(51) Int. Cl. B62J 1/00; A61F 2/50; A61F 2/80

(52) U.S. Cl. CPC B62J 1/007; A61F 2/80; A61F 2002/5006; A61F 2002/5038; A61F 2002/5083

(57) ABSTRACT

A Dynamic Prosthetic Interface (DPI) biking mount that is an external device that attaches directly to the bike of a transfemoral cyclist.

### RELATED APPLICATIONS

This application claims priority to U.S. Provisional Application No. 63/641,933 filed on May 2, 2024, which is incorporated herein in its entirety.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

Para-athletes face many challenges in all aspects of high-level competitions. Most, if not all these athletes, require completely custom equipment for optimal performances due to the nature and uniqueness of each athlete's injury. As a result, there are very little commercial options available that would make competing simpler and more efficient. Whether it be performance boosting gadgets or more comfortable options, para-athletes are not afforded the same care as other non-disabled athletes of the same caliber.

### SUMMARY OF THE INVENTION

In one embodiment, the present invention concerns a Dynamic Prosthetic Interface (DPI) biking mount. The DPI mount is an external device that will attach directly to the bike of a transfemoral cyclist. The device will serve as a high-performance and comfortable residual limb mount that will improve riding performance and increase sustained riding times.

In another embodiment, the present invention concerns a DPI mount that secures the residual limb such that the limb does not negatively affect riding performance.

In another embodiment, the present invention concerns a DPI mount that concerns a residual limb mount that ensures an easy install of the mount on multiple bikes of various configurations.

In another embodiment, the present invention concerns a DPI mount that integrates a dynamic rotational and translation system that serves to make force applications more efficient, and by improving the cyclist's comfort level during sustained riding.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 shows one embodiment of a dynamic prosthetic interface biking mount attached to a bike frame.

FIG. 2 is an isometric view of a connecting mount for an embodiment of the present invention.

FIG. 3 depicts a frame mount for an embodiment of the present invention.

FIG. 4 depicts a dampener for an embodiment of the present invention.

FIG. 5 shows components of the dampener shown in FIG. 4 for an embodiment of the present invention.

FIG. 6 depicts a rigid mount for an embodiment of the present invention.

### DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

In one embodiment, the present invention provides a DPI mount that improves the performance of a para-cyclist with a transfemoral amputation. The mount will include rotational and translational movement that not only will increase the comfort level of the rider but will allow for smoother transitions between different bikes. The mount will be made of highly durable materials that are able withstand the full limb force of the rider without damaging the mount nor the bike frame. Moreover, the mount will be easy to install and remove at the discretion of the rider, and easily transferrable to a bike of various models. All in all, the DPI bike mount will provide an enhanced experience for these high-level athletes during both casual and competitive riding.

### Embodiments of the Present Invention

All designs of the present invention are aimed to reduce the risk of catastrophic failure and injury of the cyclist when cyclical forces are applied at high riding speeds. The designs are outlined and explained below.

### Rotating Shaft with Top Tube Mounting

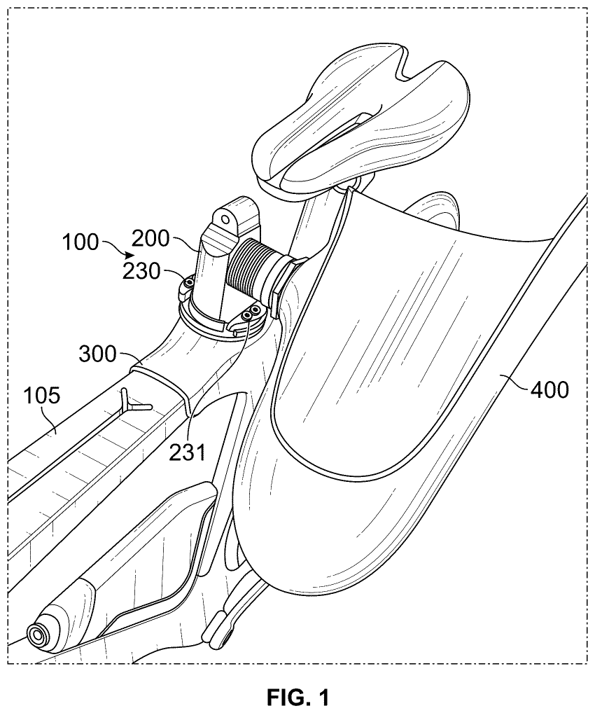

As shown in FIG. 1, DPI mount 100 for this embodiment provides the following components: frame mount 300 which is design to be removably attached to bike 105, connecting mount 200 and residual limb mount 400. Connecting mount 200 connects frame mount 300 to residual limb mount 400. The mounts provide rotational and lateral adjustability in a compact design that maintains safety and reliability.

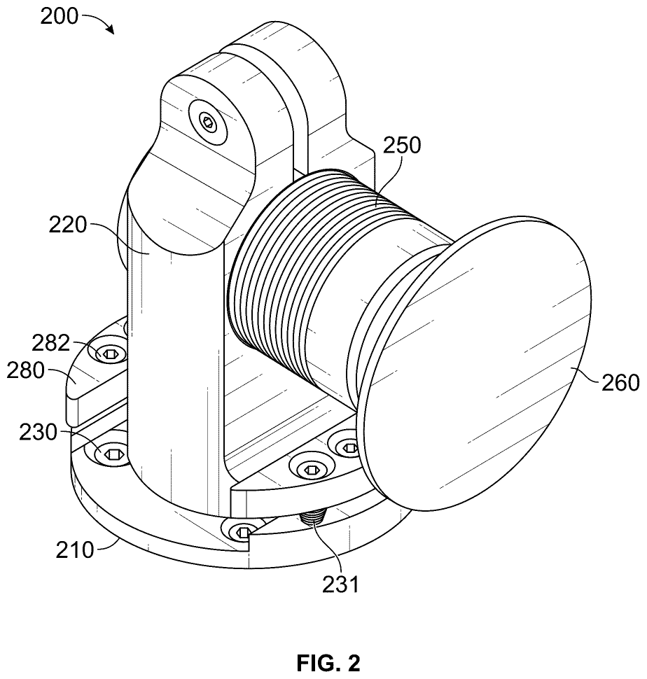

FIGS. 2 shows connecting mount 200. Connecting mount 200 includes a top tube mounting system having a pivot point comprised of plate 210 and connected post 220. Post 220 is also adapted to rotate around plate 210.

Rotating shaft 250 is connected to post 220 and has a distal end which includes member 260 to which residual limb mount 400 is mounted. In a preferred embodiment, residual limb mount 400 is rotatable when connected to connecting mount 200. This may be accomplished in several ways known to persons of ordinary skill in the art. For example, shaft 250 may include threads which engage post 220 allowing for both rotational and lateral movement or adjustment. Shaft 250 may also be rotated using bearings.

Member 260 may be threaded onto shaft 250 for both rotational and lateral movement or adjustment. As described above, bearings may be used for rotational movement.

As shown in FIGS. 1 and 2, mounting holes 230-231 may be used to attach mount 200 to bike 105. Plate 210 may include section 280 which is designed to extend a sufficient distance away from shaft 220 to allow opening 282 to receive a seat post (not shown). This permits mounting to the seat post instead of the bike frame.

### Strap Mounting

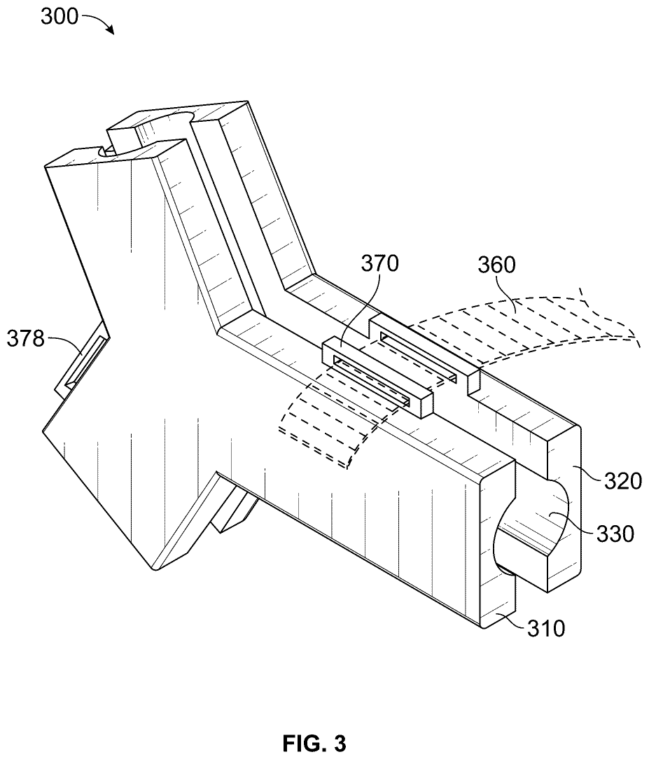

FIGS. 1 and 3 show frame mount 300 which may in the form of a shell that is designed to be affixed to bike 105. Frame 300 may include sections 310 and 320 and opening 330, which is adapted to conform to the configuration of a bike frame. In use, sections 310 and 320 are urged towards one another to create a clamping action that affixes frame mount 300 to a bike. The clamping force may be generated using fasteners, straps and other ways known to those of ordinary skill in the art.

As shown in FIG. 3, one or more straps 360 may be fed through the buckles 370 and 378 and be fastened using standard methods.

The device is attached to the bike using buckles and straps with high tensile strengths. Buckles on the backside and bottom of frame mount 300. This feature allows for the easy removal of the mount and avoids any permanent effects on the bike. Moreover, the straps can be tightened or loosened at the discretion of the user depending on application needs. The choice of straps is entirely dependent on the user and can be selected based on different factors like appearance, elasticity, material etc.

### Spring Damping Cylinder with Seat Post Mounting

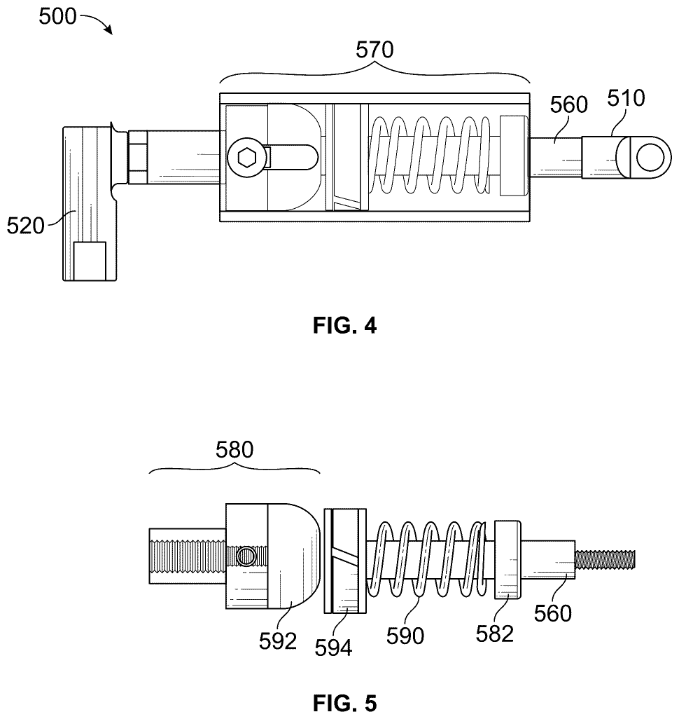

Another embodiment of the present invention incorporates a damping cylinder 500 that achieves rotation and translation as shown in FIGS. 4 and 5. In this embodiment, a mounting plate such as member 260 receives and allows clevis rod end 510 of cylinder 500 to rotate about a pivot point. The bottom of the cylinder would then be mounted to a ball joint 520 connected to a rod extending from the bicycle body. The benefit of embodiment lies mainly in the dampening of the rotational and lateral movement by a dampener 500. This design mitigates the amount of impact force generated from a hard stop.

Dampener 500 includes internal rod 560, cylinder body 570, and the end stop 580. In this design, DPI system has weather resistant using corrosion-resistant materials and a spring-loaded rotary shaft seal 582 to prevent debris from entering the cylinder body. Also included are compression spring 590, bumper 592 and wear ring 594. Wear ring 594 maintains rod stability and allow for the ring to be replaced periodically.

### Rigid Fixture

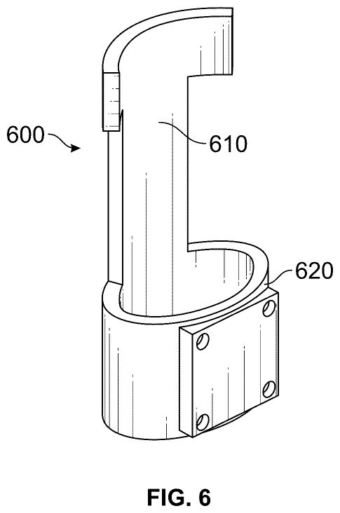

As shown in FIG. 6, another embodiment provides a rigid mount 600 adapted to attach directly to the seat post. Rigid mount 600 includes extension arm 610 to offset forces and a ring 620 that fits to the seat post. This design allows for universality in application between multiple bikes. Moreover, the device would also be easy to install (and remove), which would eliminate any potential issues that could result from more complex installation procedures. Extension arm 600 may be adapted to prevent any rotation about the seat post. This would keep the prosthetic stable when riding and allow for more efficient force application.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above-described embodiments, methods, and examples, but by all embodiments and methods within the scope and spirit of the disclosure.

What is claimed is:

1. A dynamic prosthetic interface (DPI) biking mount that is an external device that attaches directly to the bike of a transfemoral cyclist comprising:
a frame mount which is configured to be removably attached to a bike;
a connecting mount, limb receiver and residual limb mount;
said connecting mount connects frame mount to said residual limb mount; and
said connecting mount and residual limb mount provide rotational and lateral adjustability to said limb receiver with respect to a bike.

2. The dynamic prosthetic interface (DPI) biking mount of claim 1 wherein said connecting mount includes a top tube mounting system having a pivot point comprised of plate and connected post.

3. The dynamic prosthetic interface (DPI) biking mount of claim 2 wherein said connected post is adapted to rotate around said plate.

4. The dynamic prosthetic interface (DPI) biking mount of claim 3 wherein said rotating shaft is connected to said post and has a distal end which includes member to which a residual limb mount is mounted.

5. The dynamic prosthetic interface (DPI) biking mount of claim 4 wherein said residual limb mount is rotatable when connected to said connecting mount.

6. The dynamic prosthetic interface (DPI) biking mount of claim 5 wherein said residual limb mount is rotatable when connected to said connecting mount.

7. The dynamic prosthetic interface (DPI) biking mount of claim 1 wherein said frame mount is in the form of a shell that is designed to be affixed to a bike.

8. The dynamic prosthetic interface (DPI) biking mount of claim 7 wherein said frame mount includes a plurality of sections and opening which is adapted to conform to the configuration of a bike frame.

9. The dynamic prosthetic interface (DPI) biking mount of claim 7 wherein said frame mount includes a plurality of sections and opening which is adapted to conform to the configuration of a bike frame and said sections are urged towards one another to create a clamping action that affixes said frame mount to a bike.

10. The dynamic prosthetic interface (DPI) biking mount of claim 1 further including a dampener comprised of a damping cylinder connected to said mounting plate.

11. The dynamic prosthetic interface (DPI) biking mount of claim 10 wherein said dampener includes an internal rod, cylinder body, and the end stop.
